(19) United States
(12) Patent Application Publication
(10) Pub. No.: US 2013/0005812 A1
(43) Pub. Date: Jan. 3, 2013

(54) METHOD FOR ALLEVIATING ALLERGIC ASTHMA AND ALLERGIC RESPONSE BY USING BITTER MELON COMPOSITION

(75) Inventors: Bi-Fong Lin, Taipei City (TW); Ching-Jang Huang, Taipei City (TW); Hsueh-Yun Lu, Taipei City (TW)

(73) Assignee: NATIONAL TAIWAN UNIVERSITY, Taipei City (TW)

(21) Appl. No.: 13/484,716

(22) Filed: May 31, 2012

(30) Foreign Application Priority Data
Jun. 30, 2011 (TW) 100123163

(51) Int. Cl. A61K 31/201; A61P 11/06; A61P 37/08; C07C 57/12

(52) U.S. Cl. 514/560; 554/224

(57) ABSTRACT

Disclosed is a method for alleviating allergic asthma by using a bitter melon composition. The bitter melon composition comprises at least an effective amount of conjugated linolenic acid (CLN), wherein the bitter melon composition is administered to a subject suffering from allergic asthma to alleviate its symptoms. In the future, the bitter melon composition can be developed to as a health food, so as to prevent asthma, or alleviate the various symptoms of allergic asthma, such as airway hyperresponsiveness (AHR), airway inflammation, infiltration of inflammatory cell in lung tissue, or high level of serum IgE.

### BACKGROUND OF THE INVENTION

1. Field of the Invention

The present invention is related to medical application of bitter melon, especially relating to bitter melon composition for alleviation of allergic asthma symptoms and allergic response.

2. The Prior Arts

The prevalence and incidence of allergic diseases, including dermatitis, allergic rhinitis and asthma, has increased dramatically worldwide in recent years. It is generally believed that allergic diseases are related to genetic heritage, environmental pollutions, and diet habit. Allergic reaction is a response of chain reaction when human or animal contact foreign allergen. There are varieties of allergens, such as mites, pollen or small molecular proteins, which all can trigger immune response of allergic reactions. In general, allergic diseases belong to the group of Type I hypersensivity, that is mainly IgE-mediated allergic reaction, such as asthma, pollen fever and hives.

Astham, as defined in 1997 by National Heart, Lung and Blood Institute (NHLBI), is a chronic, inflammatory respiratory disease. The symptoms of asthma include periodically wheezing, cough, chest tight, even short of breath. These symptoms are often worse in the morning or at night when temperature change is significant, in contact with allergen(s) or other stimulator(s). Clinically, the main symptoms include airflow obstruction, which usually will be back to normal spontaneously or after treatment; airway hyperactivity, when contact with stimulants; and chronic airway inflammation in the long term. Because the trachea is in a persistent inflammation condition, any allergen stimulation can cause bronchial contraction which results in breath difficulty, called airway hyperresponsiveness (AHR).

In the past forty years allergic diseases have increased dramatically worldwide. According to the WHO estimation done in 2005, about three billion people feel discomfort due to asthma worldwide, and it is estimated that 250 thousands of people die of asthma. In Taiwan prevalence and incidence of asthma among children and teenagers have increased significantly in the past 20 to 30 years. For example, asthma prevalence has increased from 1.3% in 1973 to 19.0% in 2003.

Currently common treatments of allergic diseases are direct administration of bronchial dilator or anti-inflammatory drug, such as anti-histamine, β-adrenergic agonist and glucocorticoid, which mainly targeting for relief of asthma symptoms (Barnes et al., 1997; Holgate and Broide, 2003), but these types of treatment show no therapeutic effects. In addition, there are treatments that use monoclonal antibody as inhibitor of cell activity, or oral administration of allergen to build up a subject's tolerance of immune response for relief of hyperallergic reactions. Bronchdial dilator and steroid are commonly used medications, in which prednisolone is the most common oral steroid administered clinically. However, medication may accompany side effects.

It is currently known that bitter melon in the diet is beneficial to human health, including decrease of blood sugar. Especially, high amount of special fatty acids in the bitter melon, such as conjugated linolenic acid (CLN), are potential active compound. However, it has not been confirmed whether the bitter melon and its active compound mentioned above can be applied as medication for alleviation of allergic asthma, and the effect of bitter melon on immuno-regulation and inflammatory reaction has not been studied. Therefore, it is urgent to confirm that easily obtainable foods or their active ingredients, such as bitter melon, can be consumed by individuals with genetically allergic physique to alleviate allergic asthma without well-known side effect of medicine.

### SUMMARY OF THE INVENTION

Recent epidemiology studies have shown that in addition to genetic and environmental factors, diet may also be one of the risk factors of asthma. Increase intake of antioxidant and n-3 poly-unsaturated fatty acids, or decreased ingestion of n-6 poly-unsaturated fatty may reduce occurrence of asthma, suggesting that diet does regulate allergic asthma. Common symptoms of allergic asthma include airway blockage during breathing, hyper-reaction of respiratory tract, chronic respiratory tract inflammation and Th2 immune reaction. Currently there are some commercially available healthy foods claiming to relief of allergic symptoms. Thus, conveniently available foods have the potential being developed as nutraceuticals to regulate body immune system to alleviate symptoms of allergic asthma and to minimize side effect of medications.

Therefore, one objective of the present invention is to provide a method for alleviating allergic asthma and allergic response by administrating a bitter melon composition to a subject suffering from allergic asthma.

The other objective of the present invention is to provide a bitter melon composition for alleviating allergic asthma.

The present invention is related to a method for alleviating allergic asthma and allergic response, comprising administrating a bitter melon composition to a subject suffering from allergic asthma, wherein the bitter melon composition comprises at least an effective amount of conjugated linolenic acid (CLN).

Moreover, the present invention provides a bitter melon composition for alleviating allergic asthma, wherein the bitter melon composition alleviates a subject's allergic asthma symptom.

The present invention uses the asthma mouse model to demonstrate effects of ingestion of bitter melon and conjugated linolenic acid on inflammatory and allergic immune response, in which assessed factors including inflammatory response of local respiratory tract, airway resistance, systemic immune response and gene expression in lung tissue. In the examples of the present invention, freeze-dried bitter melon powder were fed to the mouse model of allergic asthma sensitized with ovalbumin (OVA, albumin chicken egg, Sigma A-5378), observation of alleviation of asthma symptoms are profound, including reduction of airway hyperresponsiveness, AHR), reduction of airway eosinophil infiltration, decreased cytokine secretion of IL-4, IL-5, IL-13, and IL-6 in bronchoalveolar lavage fluid (BALF). Furthermore, bitter melon seeds are ample of conjugated linolenic acid (CLN). Results of the present invention demonstrate that improvement of asthma symptoms, including decreased airway hyperresponsiveness, reduction of airway eosinophil infiltration, and decreased cytokine secretion of IL-5 in bronchoalverlar lavage, are significantly in mouse model of allergic asthma fed with pure CLN.

In the practice of the present invention, freeze dried bitter melon powder (BGP) and conjugated linolenic acid (CLN) are used to feed mouse model of allergic asthma challenged with ovalbumin The results demonstrate that administration of diet containing 5% of BGP or tube feeding with 35 mg of CLN daily can alleviate respiratory tract symptoms of asthma and suppress allergic response in sensitized BALB/c mice after 16 days. Effects of BGP on symptom relief include (1) decrease of airway hyperresponsiveness; (2) reduction of the amount of IL-4, IL-5, IL-13, IL-6, eotaxin and prostaglandin E₂ (PGE₂) (Table 3); (3) inhibition of secretion of IL-13 of splenocyte OVA-specific IL-13; (4) reduction of amount of IgE in serum and increase amount of IgG in the serum; and (5) increase of expression of PPAR-α mRNA in lung tissue. Mice fed with CLN showed alleviation of symptoms described above in 1 to 3.

The results demonstrate that freeze dried bitter melon powder containing CLN has the efficacy to alleviate allergic asthma. In the future, the BGP has the potential to be developed as nutraceuticals to prevent asthma or to alleviate symptoms of asthma, and the BGP can be either an ingredient or as single component of food for improvement of asthma symptoms. The present invention can be in the form of food or ingredient in single effective dosage, or further containing an additive. The additive can be an ingredient of healthy food, a food ingredient or a combination of healthy food ingredient and food composition, to alleviate asthma symptoms induced by allergy, such as airway hyperresponsiveness, airway inflammation, infiltration of inflammatory cells in the lung tissue, and amount of IgE in the serum.

### BRIEF DESCRIPTION OF THE DRAWINGS

FIG. 1 exhibited sensitization and feeding program of mouse model of allergic asthma.

FIG. 2 showed effects of diet supplemented with BGP and CLN on airway hyperresponsiveness of OVA-sensitized mice.

FIGS. 3A˜3B demonstrated effects of diet supplemented with BGP and CLN on cell numbers of different cell type in BALF of OVA-sensitized mouse model of allergic asthma.

FIGS. 4A˜4B showed effects of diet supplemented with BGP on the level of total antibodies in serum of OVA-sensitized mouse model of allergic asthma.

FIG. 5 exhibited effects of diet supplemented with of BGP on PPARα mRNA expression in OVA-sensitized mouse model of allergic asthma.

### DETAILED DESCRIPTION OF THE PREFERRED EMBODIMENT

The present invention of preparation and application of bitter melon composition in alleviation of allergic astham is further explained in the following embodiment illustration and examples. Those examples below should not, however, be considered to limit the scope of the invention, it is contemplated that modifications will readily occur to those skilled in the art, which modifications will be within the spirit of the invention and the scope of the appended claims.

The present invention uses mouse model of allergic asthma to investigate effect of ingestion of bitter melon and conjugated linolenic acid on airway inflammation, the assessed parameters include inflammation reaction of local respiratory tract, airway resistance, systemic immune response, and changes in gene expression of lung tissue.

### Materials and Methods

### 1. Preparation of Freeze Dried Bitter Melon Powder or Source of CLN

The bitter melon used in the present invention is Hualien No. 4 bitter melons, developed, cultivated and provided by Hualien District Agricultural Research and Extension Station, Council of Agricultural, Executive Yuan, Taiwan. Fresh bitter melon was washed, sliced, freeze dried, and then minced with a grinder into a bitter melon powder (BGP). Mice were fed with diet containing 5% of BGP in animal tests. Conjugated linolenic acid (c9,t11,t13-CLN) was purchased from Cayman with purity higher than 98%.

### 2. Design of Animal Experiment

### (1) Feeding of Mouse Model

Inbred female BALB/c mice (4 weeks old) were purchased from the Laboratory Animal Center, National Taiwan University and housed in a room with constant temperature of 23±2° C. The animal room was kept on a 12-h light and 12-h dark cycle. The mice were housed and kept on free diet and water to acclimatize before feeding the experimental diet. Diet ingestions are recorded every 3˜4 days, and mice body weight were recorded every week. When mice were in 8 week old, the mice were sensitized and challenged to induce allergic reaction at week 8, 10 and 12 by an intraperitonal injection (i.p.) of antigen containing ovalbumin antigen and adjuvant (Imject® Alum). Blood samples were collected on two days before injection and on two weeks after last injection for analysis of OVA-specific antibodies. Mice were divided in five groups based on OVA-specific IgE antibody and body weight, namely OVA-oil group (the control group), OVA-BGP group (feed containing 5% of freeze dried bitter melon powder), OVA-CLN group (tube feeding containing 35 mg CLN), OVA-Pred (prednisolone, the positive control group), and PBS-oil (the non-sensitized group), respectively. Mice diet was based on AIN-76 formulation (feed composition referred to J. Nutri. 107: 1340-1384, 1977). Administration of steroid prednisolone by tube feeding started 4 days before AHR induction and last to the day sacrificed. The feed formulation and tube feeding formulation was shown in Table 1a-1b. After 4 days of feeding, mice were challenged with 5% aerosol of OVA solution, while the PBS-oil group was administered with aerosol PBS. Change of mice airway resistance was measured after 13 days of feeding, and mice were sacrificed on day 16.

### (2) Sensitization Method

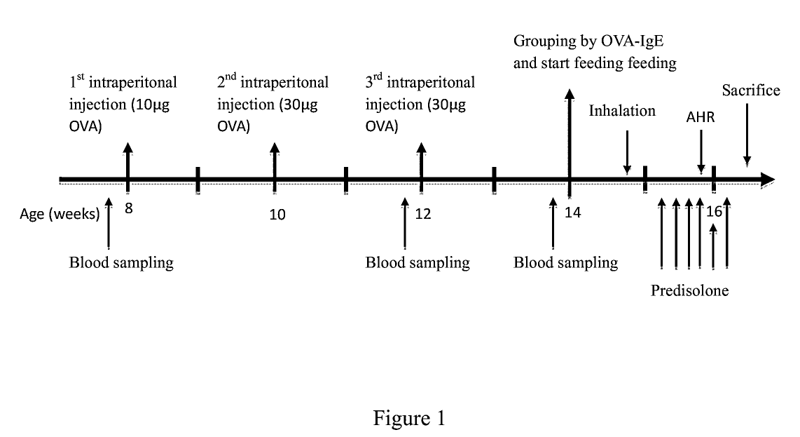

The sensitization method uses OVA as antigen, emulsified in a adjuvant of Al(OH)₃. When mice were in equilibrium period (8 week old), the mice were sensitized and challenged to induce allergic reaction at week 8, 10 and 12 by an intraperitonal injection (i.p.) of alum-precipitated antigen containing ovalbumin antigen as described. The PBS-oil group uses heat-treated PBS to replace OVA. Each mouse received a dose of 0.2 ml. During group feeding, the day before airway resistance testing and the day before sacrifice, mice were challenged by 5% aerosolized OVA to induce airway allergic inflammatory reaction. The PBS-oil group is challenged with PBS, which was to replace the OVA. FIG. 1 showed the mouse sensitization protocol.

### (i) Sensitization by OVA Intraperitonal Injection (i.p.)

Mice were first sensitized by an intraperitonal injection (i.p) of antigen [at a concentration of OVA (10 μg/mice) and Al(OH)₃ (2 mg/mice) dissolved in 0.2 ml PBS]. For the second and third sensitization, mice were injected with antigen at a concentration of OVA (30 μg/mice) and Al(OH)₃ (2 mg/mice) per 0.2 ml PBS.

Antigen: Ovalbumin (OVA, albumin chicken egg, Sigma A-5378) was prepared to final concentration of 20 mg/ml and stored at −20° C.

(ii) (Adjuvant): Imject® Alum, 40 mg/mL

Adjuvant: Imject® Alum, 40 mg/mL

(iii) OVA Inhalation (i.h) Sensitization

Mice were challenged by aerosol of OVA at concentration of 50 mg/ml, dissolved in Phosphate-buffered saline (PBS) for 30 min. The aerosolized OVA were created using an ultrasonic nebulizer (Ultra®99) with controlled flow rate at 0.4 ml/min.

TABLE 1a
Diet formulation (feeding unit: g/kg)

| | AIN-76 | | |
|---|---|---|---|
| Composition | Normal | 5% BGP | |
| Casein (ICN, USA) | 200 | 197.8 | 2.2 |
| Methione (Sigma, M9500) | 3 | 3 | |
| Cereal Starch *Samyang genex, Korea) | 150 | 122.7 | 27.3 |
| Sugar (Taiwan Sugar fine sugar, Taiwan) | 500 | 500 | |
| Cellulose (JRS, Germany) | 50 | 30.9 | 19.1 |
| Soybean oil (Taiwan Sugar) | 50 | 48 | 2 |
| AIN-76 Vitamin Mix¹ (ICN) | 10 | 10 | |
| AIN-76 Mineral Mix¹ (ICN) | 35 | 35 | |
| Choline (Sigma, C1879) | 2 | 2 | |
| Bitter melon powder | — | 50 | |

Note:
¹Composition of AIN-76 Vitamin mix and AIN-76 mineral mix is described in J. Nutri. 107: 1340-1384 (1977)

TABLE 1b
Composition of tube feeding and diet

| Group | Tube feeding | Diet |
|---|---|---|
| OVA-oil | 0.1 mL soybean oil | AIN-76 |
| OVA-BGP | 0.1 mL soybean oil | AIN-76 |
| | | containing 5% BGP |
| OVA-CLN | 35 mg CLN in 0.1 ml Soybean oil | AIN-76 |
| | (1% CLN) | |
| OVA-Pred | 2 mg pred/kg BW in 0.1 ml soybean | AIN-76 |
| | oil | |
| PBS-oil | 0.1 ml soybean oil | AIN-76 |

### 3. Various Immune Parameter Determination

### (1) Airway Hyperresponsiveness (AHR)

After an inhaled antigen challenge, airway resistance of the mice were tested the following day using Buxco system (Biosystem XA, Buxco Electronics Inc. Sharon, Conn., USA), in which the differential pressure transducer (Buxco) and preamplifier (MAX II, Buxco) were used to collect change information of mice respiratory and to calculate lung Penh value (Penh, enhanced pause). Mice were placed into whole-body plethysmograph chamber, and aerosol of PBS was introduced into the chamber for 3 mins by using an ultrasonic nebulizer. After excess aerosol was evacuated, an average Penh value per minute was recorded. Then mice were challenged again with elevated concentration of methacholine (6.25, 12.5, 25, 50 mg/ml) aerosol. The same challenge process was performed at each concentration, and an average Penh value per minute was recorded after 3 mins after each challenge.

Penh value was calculated as Penh=Pause×PIF/PEF; wherein Pause=(Te−Tr/Tr); PIF: peak inspiratory flow; PEF: peak expiratory flow; Te: expiratory time; Tr: relaxation time.

### (2) Collection and Treatment of Bronchoalveolar Lavage Fluid (BALF)

After mice were sacrificed, fur and skin in the mice neck were incised and muscle surrounding the trachea was tear open to expose the trachea. A small hole was made on the trachea near the head with scissors to intubate tracheal tubing. After intubation, their lungs and airways were lavaged with 0.5 ml of sterile PBS. Two ml of bronchoalveolar lavage fluid (BALF) was centrifuged at 1500 rpm for 7 mins at 4° C. The supernatant (BALF) was collected and stored at −80° C. for cytokine concentration determination. The cell pellet was washed with HBSS buffer twice and re-suspended in 10% FBS/RPMI medium. Total leukocytes were counted with a hemocytometer using trypan blue dye exclusion method. After the cell concentration was adjusted to 5×10⁵ cell/ml, aliquots of 100 μl total cells were centrifuged with cytospin at 500 rpm for 2 mins and then stained with Liu A for 30 seconds. After excess stain was washed off and air dried, the cells were stained with Liu B for 5 seconds. Slides were air-dried and sealed with Arabic gum. The cells were observed with a microscope (Olympus, BX41 TF, Japan) equipped with an oil immersion lens under 1000 times of magnification. Two hundreds of leukocytes were counted on each slide, which includes four different cells, eosinophil, neutrophil, lymphocyte and monocyte.

### (3) Serum Collection and Determination of Antibody in Serum

### (i) Collection of Blood Sample

Blood samples of mice were collected at designated time. Mice were placed in a chamber and anesthetized with diethyl ether, bled using retroorbital venous plexus puncture to collect blood (about 100 μl). Blood samples were stored at 4° C. for 3 to 4 hrs and then centrifuged at 12000 rpm for 20 mins to collect serum. Serum samples were stored at −80° C. until use.

### (ii) Determination of OVA-Specific IgE, IgG1, IgG2a in the Serum

Aliquots of 200 μl/well OVA (10 μg/ml, dissolved in coating buffer) were pipeted into the 96 wells microplate (Nunc-Immuno plate). Plates were incubated overnight at 4° C. Then, the plates were carefully washed with PBST four times to remove un-binding monoclonal antibody and then aliquots of 200 μl/well blocking buffer were pipeted into the 96 microplate wells to block the free sites on the wells and minimize non-specific binding. After incubation for 2 hours at room temperature, the plates were washed with PBST buffer five times then aliquots of 100 μl/well diluted serum samples or known concentration standards were pipeted into the 96 microplate wells, respectively. The OVA specific IgE plates were incubated overnight at 4° C. while the OVA specific IgG1 or IgG2 plates were incubated for 2 hours at room temperature. Plates were washed with PBST six times and aliquots of 100 μl/well biotin conjugated cytokine secondary antibody were added. After incubation for 2 hours at room temperature, the plates were washed with PBST buffer seven times then aliquots of 100 μl/well avidin-peroxidase were added. After incubation for 2 hours at room temperature, the plates were washed with PBST buffer eight times then finally aliquots of 100 μl/well substrate TMB were added. After proper incubation to develop the color, the plates were read on an ELISA plate reader at 620 nm and optical densities were converted into arbitrary ELISA units (EU). In this experiment, pooled sera from previously sensitized mouse were served as positive controls (Dilution fold of OVA-specific IgE, OVA-specific IgG1, and OVA-specific IgG2 was 50-fold, 160,000-fold, and 8.000-fold, respectively). The E.U. was calculated according to the formula=(ODₛₐₘₚₗₑ−OD_{blank})/(OD_{PC}−OD_{blank}).

(iii) Serum Total IgE, IgG, IgA and IgM Concentration Determination

96-well plates were coated with 10 ng/ml (100 μl/well) affinity-purified antibody, allowed to incubate at room temperature for one hour, and then washed with TBST four times. Blocking solution (200 μl/well) was added to the plates, incubated for one hour at room temperature, and then washed with TBST for five times. The serum samples (100 μl/well) and standards were appropriately diluted and added to the 96-well plate, respectively. After 2 hour incubation, the plates were washed six times and HRP-conjugated affinity purified antibody (100 μl/well) were added. After further one hour incubation, plates were washed with TBST seven times. Finally, enzyme substrate solution TMB (100 μl/well) and the plates incubated in the dark. The antibody levels of the samples were determined after color developed by measurement of absorption at 620 nm

### (4) Collection and Culture of Splenocytes

Mice were sacrificed and their spleens were aseptically removed and suspended in 3 cm incubation dish containing 3 ml TCM/RPMI solution. Spleen tissue was minced with rear end of syringe and the upper layer cell suspension was collected and placed in 15 ml centrifuge tube. Another 3 ml HBSS buffer was added to wash the dish and residual cells were collected. Cell suspensions were centrifuged at 1500 rpm for 10 mins Supernatant was discard and the precipitated cells were re-suspend in ACK lysing buffer. After 1 minute, HBSS buffer was added to the suspension and then centrifuged at 1500 rpm for 10 mins Cell pellet was washed with HBSS buffer for 3 times. 2 ml of TCM/RPMI medium was added to re-suspend the cells. Cell number was counted and adjusted to 1×10⁷ cell/ml. 500 μl of the cell suspension was added to 48 well plates. TCM/RPMI culture media containing ConA were added to the plate to adjust the cell density at 5×10⁶ cell/ml and ConA final concentration at 1.25 μg/ml. After 48 hours incubation, supernatants were collected and stored at −80° C.

### (5) Determination of Cytokine Concentration

BD Pharmingen System was provided as an example. Cytokine concentration was determined by ABC (avidin-biotin conjugated system) ELISA method. Primary antobiody was added to the 96-microwell plate wells and incubated at 4° C. overnight. After incubation, plates were washed four times with PBST buffer. To block non-specific binding, block buffer were added to each well and the plates were incubated at room temperature for 1 h. After incubation, plates were washed five times with ELISA wash buffer. Appropriately diluted samples or standard solution were then added to the 96-microwell plate wells and the plates were incubated at room temperature for 2˜3 h. After incubation, plates were washed with buffer six times then secondary antibody for detection was added to each well. Plates were incubated for another 1 h. After incubation, plates were washed seven times with ELISA wash buffer. Streptavidin-HRP (horseradish peroxidase) was added to each well and the reaction was allowed to react for 30 mins. After reaction, the plates were washed with ELISA buffer eight times and then HRP-substrate solution was added to the 96-microwell plate wells. Plates were incubated at room temperature for 20˜30 mins to develop color. The plates were measured the absorbance at 405 nm by ELISA reader.

### (6) Real-Time PCR Analysis of Lung Tissue Genes

### (i) Extraction of Total RNA

1 ml TRIZOL Reagent (Invitrogen) was added to the right lung tissue and homogenized on ice with electric homogenizer. The homogenate was transferred to 1.5 ml micro-centrifuge tube, placed at room temperature for 5 mins and centrifuged at 12,000 rpm for 20 mins at 4° C. The pink layer solution was collected and transferred to a new 1.5 ml centrifuge tube and 0.2 ml chloroform (Merck) was added. The solution was shook well and placed at room temperature for 2 to 3 mins to perform phase separation and then centrifuged at 12,000 rpm for 15 mins at 4° C. Upper aqueous layer was collected and transferred to a new micro-centrifuge tube. 0.5 ml of isopropanol (Merck) was added, vortex and incubate the sample at 4° C. for one hour and then centrifuged at 12,000 rpm for 20 mins. The upper supernatant was removed. 0.4 ml DEPC-H₂O was added to the pellet. Vortex until the RNA pellet dissolved. Equal volume of DEPC-H₂O saturated phenol (Amreso 0981) was added, vortex for 30 seconds, and then centrifuged at 12,000 rpm for 5 mins at 4° C. Upper aqueous layer was collected, and 0.4 ml chloroform was added. The solution was vortex for 30 seconds and then centrifuged at 12,000 rpm for 20 min at 4° C. Collect Upper aqueous layer was collected and 0.1-fold volume of 3M sodium acetate (pH 5.2) and 2-fold volume of absolute ethanol were added. Solution was well mixed and stored at −20° C. to precipitate RNA for 1 hour (or overnight precipitation to increase yield). The solution was centrifuged at 12,000 rpm for 20 mins at 4° C. Upper layer supernatant was discarded, and the RNA pellet was wash twice with 70% ethanol. The pellet was air dried at room temperature and then dissolved with 20 ml of DEPC-H₂O. To determine the concentration of RNA, a small amount of RNA was diluted 100 fold and the concentration of RNA was determined at 260/280 nm, according to the formula 1 unit of 260 nm=40 μg RNA/ml.

(ii) Reverse Transcription of Total RNA to cDNA

RNA solution was diluted with DEPC solution to concentration of 0.2 μg/μL. 10 μl RNA solution, 10×RT buffer (2 μl), 25× dNTP mixture (0.8 n1), 10×RT random primer (2 n1), sterile distilled water (4.2 n1) and RTase (1 μl) was sequentially added to the 10 n1 of RNA solution to make a final 10 n1 mixture solution for reverse transcription. The reverse transcription was conducted at 25° C. for 10 mins, 37° C. for 120 mins, 85° C. for 5 seconds, and then finally cooled at 4° C. cDNA samples were stored at −20° C.

(iii) Real-Time PCR Analysis

Commercial available Taqman Universal PCR Master Mix was used and GAPDH was used as internal control. cDNA (10 μL) was properly diluted with sterile water to 10 ng/ml. 12.5 ml of Tagman Universal PCR Master Mix, 1.25 ml of probe/primer mixture and 1.25 ml sterile water to make final 25 n1 mixture. ABI PRISM Optical Strip was added and covered with MicroAmp Optical cap. After removal of air bubble with a brief centrifugation process, the PCR mixture was ready for quantitative analysis using ABI PRISM 7000 Sequence Detection. The reaction condition was set at 50° C. for 2 mins (stage 1), 95° C. for 10 mins (stage 2), 95° C. for 15 seconds (stage 3), and 60° C. for 1 minute (stage 4), and then back to stage 3. The cycle was repeated for 40 times.

### Calculation

Ct value (Threshold Cycle): C represents Cycle number, T represents threshold. Ct value (threshold cycle) is the cycle number at which the fluorescence generated within a reaction crosses the fluorescence threshold, a fluorescent signal significantly above the background fluorescence. The threshold cycle is inversely proportional to the original relative expression level of the gene of interest. PCR product is linearly proportional to the quantity of initial cDNA. Ct value is inversely proportional to the log concentration of input cDNA. When amount of input cDNA is fixed, the amount of the product is proportional to 2′.

Step 1. Ct (target gene)−Ct (internal control)=ΔCt

Step 2. ΔCt (Sample)−ΔCt (mean of control)=ΔΔCt

Step 3. Amount of gene expression is presented as 2^{-ΔΔCt}.

### (7) Statistic Analysis

All data were presented as mean±SD or mean±SEM. All analysis for statistically significant differences was performed with Student's t-test as compared with treatment to the OVA-oil group. p-values <0.05 were considered significant. *p<0.05, ** p<0.01, ^{#}0.05<p<0.1.

### Results

### Example 1

### Growth and Diet of Mice

BALB/c mice were sensitized every other week for three times. Two weeks before the last sensitization mice were divided into five groups according to OVA-specific IgE and body weight, then different experimental diet were provided for 16 days. There was no significant difference of body weight among five groups of mice before feeding and when sacrificed (Table 2), indicating freeze dried bitter melon powder (BGP) or CLN diet did not affect mice growth. In the aspect of daily ingestion, no difference was observed among these mice groups.

TABLE 2
The effects of BGP and CLN on growth and feeding of
OVA-sensitized mice

| | | Initial Body | Final Body | |
|---|---|---|---|---|
| | | weight (g) | Weight (g) | Feed Intake |
| | n | 14 week-old | 16 week-old | (g/day) |
| OVA-oil | 9 | 21.7 ± 2.15 | 21.6 ± 1.97 | 2.96 ± 0.42 |
| OVA-BGP | 9 | 22.2 ± 2.26 | 21.3 ± 1.65 | 2.81 ± 0.30 |
| OVA-CLN | 7 | 22.7 ± 1.30 | 22.0 ± 1.52 | 2.99 ± 0.31 |
| PBS-oil | 6 | 22.1 ± 1.44 | 21.5 ± 2.12 | 2.96 ± 0.35 |

Note:
Each value represents mean ± SD.

### Example 2

### Organ Weight and Relative Weight

Effects of diet supplemented with BGP diet and CLN on various organ absolute weight and relative weight of mice were exhibited in Table 3. After 16 days of feeding, the absolute weight of heart of OVA-BGP treated mice was less than the OVA-oil treated mice. There was a trend of less absolute weight of the liver (p=0.08) in OVA-BGP treated mice, whereas the relative weight of the liver of OVA-BGP treated mice was significantly low than the control groups. There was no difference between the OVA-CLN group and control group. There was a trend (p=0.06) of lung weight in OVA-Pred group mice than the OVA-oil control group, and there was a trend of absolute weight of liver, whereas the relative weight was significant lower. The absolute weight and relative weight of lung of PBS-oil mice group were lower than sensitized mice groups.

TABLE 3
The effects of BGP and CLN on organ weight and relative organ weight of
OVA-sensitized mice

| | n | Lung | Heart | Liver | Kidney | Spleen |
|---|---|---|---|---|---|---|
| | | Absolute weight (g) | | | | |
| OVA-oil | 9 | 0.46 ± 0.06 | 0.13 ± 0.02 | 1.26 ± 0.13 | 0.29 ± 0.02 | 0.090 ± 0.014 |
| OVA-BGP | 9 | 0.45 ± 0.07 | 0.11 ± 0.01* | 1.14 ± 0.15^{#} | 0.28 ± 0.03 | 0.082 ± 0.015 |
| OVA-CLN | 7 | 0.48 ± 0.08 | 0.12 ± 0.02 | 1.28 ± 0.15 | 0.29 ± 0.01 | 0.080 ± 0.026 |
| PBS-oil | 6 | 0.30 ± 0.05** | 0.16 ± 0.09 | 1.30 ± 0.22 | 0.28 ± 0.03 | 0.082 ± 0.018 |
| | | Relative weight (%) | | | | |
| OVA-oil | 9 | 2.12 ± 0.26 | 0.58 ± 0.10 | 5.85 ± 0.33 | 1.35 ± 0.11 | 0.42 ± 0.05 |
| OVA-BGP | 9 | 2.10 ± 0.24 | 0.50 ± 0.05* | 5.34 ± 0.50* | 1.30 ± 0.12 | 0.38 ± 0.05 |
| OVA-CLN | 7 | 2.21 ± 0.46 | 0.56 ± 0.06 | 5.80 ± 0.38 | 1.30 ± 0.05 | 0.37 ± 0.12 |
| PBS-oil | 6 | 1.39 ± 0.30** | 0.56 ± 0.11 | 6.00 ± 0.77 | 1.28 ± 0.08 | 0.38 ± 0.07 |

Note 1:
Each value represents mean ± SD
Note 2:
*p < 0.05;
**p < 0.01;
^{#}0.05 p < 0.1 is the results of comparison with the OVA-oil group by Student's t-test.

### Example 3

### Airway Hyperresponsiveness

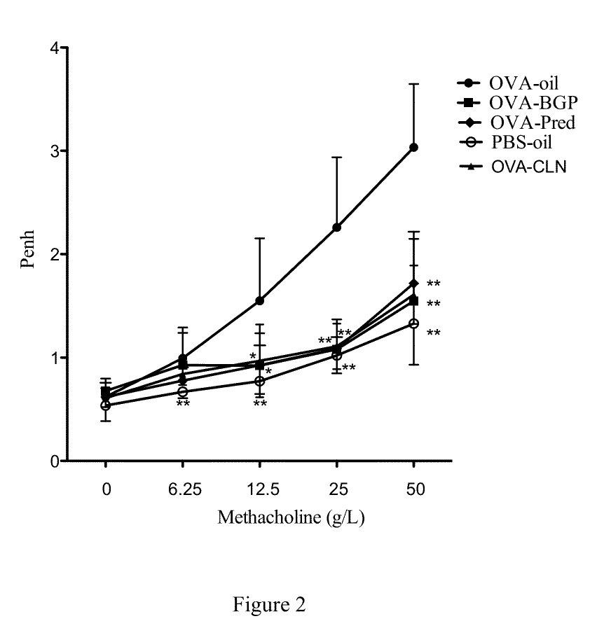

Effect of diet supplemented with BGP and CLN on AHR of OVA-sensitized mice was shown in FIG. 2. The results indicated that as the concentration of Mch increased, Penh value of the negative control mice (un-sensitized PBS-oil group) increased slowly and was the lowest among all groups (significantly lower as compared to OVA-oil group), suggesting that sensitization challenge did increase airway resistance. As compared to the OVA-oil group, mice given predisolone showed alleviation of AHR when challenged with Mch at concentration higher than 12.5 mg/ml. At the same concentration (12.5 mg/ml) of Mch challenge, mice fed with BGP and CLN also exhibited alleviation of AHR and the level of alleviation was equivalent to the drug control group (OVA-Pred group). The results indicated that AHR reaction could be significantly alleviated when mice were fed with diet containing BGP or CLN after 13 days feeding period.

### Example 4

### Changes of Assessed Factors in BALF

Effect of various treatments on change of cytokines in BALF was shown in Table 4. The IL-4, IL-5, IL-13, IL-6, Eotaxin and PGE₂ concentration decreased in OVA-BGP group.

Effect of various treatments on change of cytokines in BALF was shown in Table 4. Un-sensitized group (the PBS-oil group) had the lowest level of IL-4 and IL-5. Mice fed with diet containing BGP had significantly lower level of IL-4, IL-5 and IL-13 in BALF, whereas OVA-CLN group showed low level of IL-5 and a trend of decreased IL-4 level, suggesting that Th2 cytokine level in respiratory tract could be reduced when mice fed with BGP and CLN.

Effect of BGP and CLN supplementing diet on level of inflammatory factors in BALF was shown in Table 4. Levels of IL-6, IL-1β, Eotaxin and PGE₂ in PBS-oil group mice were low. As compared to OVA-oil group, levels of IL-6, Eotaxin and PGE₂ were lower in OVA-BGP treated mice and decrease trends of TNF-a and IL-1β level were observed. The level of TNF-α and IL-1β were also low in OVA-BGP group. Mice fed with diet supplemented with CLN also showed lower inflammatory factors as compared to OVA-oil group, but there was no statistical difference. These results indicated that diet supplemented with bitter melon could reduce levels of inflammatory factors, including IL-4, IL-5, IL-13, IL-6, Eotaxin, and PGE₂ in the BALE

TABLE 4
The effects of BGP and CLN on the level of cytokine and
eotaxin in BALF of OVA-sensitized mice

| | | IL-4 | IL-5 | IL-13 | PGE₂ |
|---|---|---|---|---|---|
| | n | (pg/mL) | (pg/mL) | (pg/mL) | (ng/mL) |
| OVA- | 9 | 74.2 ± 28.7 | 744 ± 444 | 348 ± 122 | 34.6 ± 27.1 |
| oil | | | | | |
| OVA- | 9 | 36.0 ± 29.1* | 230 ± 155** | 196 ± 85** | 12.4 ± 9.0* |
| BGP | | | | | |
| OVA- | 7 | 39.1 ± 33.9^{#} | 326 ± 183* | 254 ± 75 | 17.2 ± 16.1 |
| CLN | | | | | |
| PBS- | 6 | 5.5 ± 8.6 | 1 ± 2 | 537 ± 284 | 1.2 ± 0.3** |
| oil | | | | | |

| | | IL-6 | TNF-α | IL-1β | Eotaxin |
|---|---|---|---|---|---|
| | n | (pg/mL) | (pg/mL) | (pg/mL) | (pg/mL) |
| OVA- | 9 | 34.4 ± 35.2 | 72.5 ± 12.4 | 27.9 ± 15.6 | 28.5 ± 6.8 |
| oil | | | | | |
| OVA- | 9 | 5.8 ± 17.4* | 48.8 ± 30.8^{#} | 15.3 ± 13.2^{#} | 19.8 ± 7.4* |
| BGP | | | | | |
| OVA- | 7 | 12.2 ± 27.8 | 64.5 ± 29.3 | 21.0 ± 9.5 | 27.3 ± 8.5 |
| CLN | | | | | |
| PBS- | 6 | 0.0 ± 0.0 | 94.9 ± 65.3 | 12.7 ± 5.2 | 10.4 ± 2.6 |
| oil | | | | | |

Note 1:
Each value represents mean ± SD
Note 2:
*p < 0.05;
**p < 0.01;
^{#}0.05 < p < 0.1 is the results of comparison with the control group by Student's t-test.

### Example 5

### Leukocyte Numbers in BALF

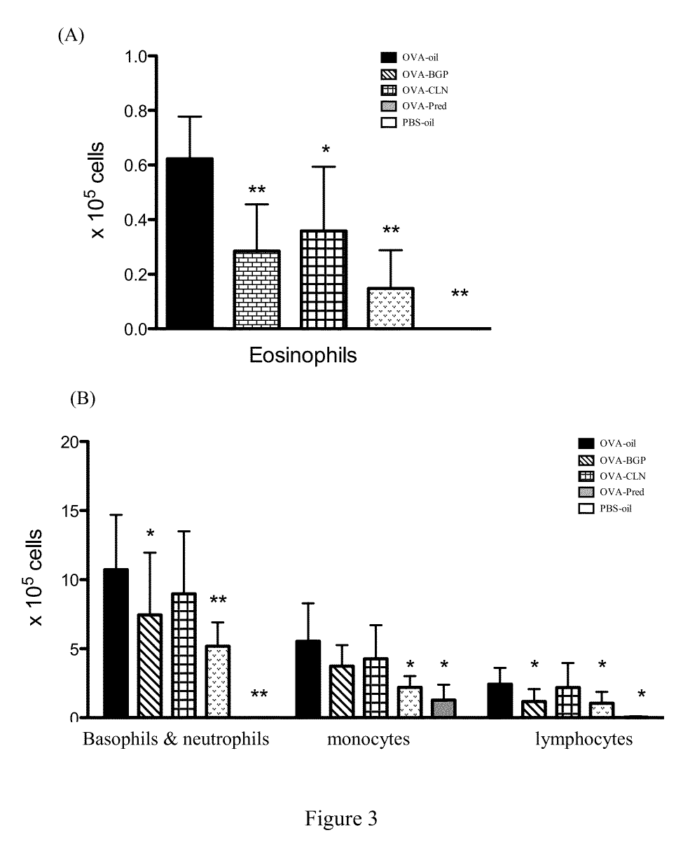

Effects of diet treatment on leukocyte number in the BALF of OVA sensitized mice were exhibited in FIG. 3A-3B. Total leukocyte number and individual leukocyte number, including eosinophils, basophils, neutrophils, macrophage and lymphocytes, and monocytes number, were significantly less in the PBS-oil group. Total leukocyte numbers and individual leukocyte number in the OVA-Pred group were also less than those in the OVA-oil group, suggesting that the medication could suppress immune cell numbers or infiltration. Mice fed with diet containing BGP showed less total leukocyte number, eosinophils, basophils, neutrophils, and lymophocyte numbers in the BALF, while the OVA-CLN group mice exhibited less eosinophils infiltration. The results indicated that BGP and CLN diet could alleviate cell infiltration in the lung, especially alleviating the eosinophils infiltration found in allergic airway inflammation.

### Example 6

### Amount of Cytokine Secretion in Splenocytes

Mice challenged with OVA were fed with diet containing BGP and CLN. The effect of BGP and CLN diet on splenocytes of OVA-sensitized mice was shown in Table 5. The IL-2 secretion in splenocytes of PBS-oil treated mice was significantly lower as compared to OVA-oil group. Same results were observed in secretion level of IL-5 and IL-13. The results suggested that un-sensitized mice tend to secrete less cytokines, while sensitized mice tend to show Th2 systemic immune response.

However, diet supplemented with BGP could suppress IL-13 secretion capability of splenocytes stimulated with OVA, in the meantime, the secretion of TGF-β also decreased. The OVA-Pred group exhibited a trend of decrease secretion of IL-5 and IL-13 (p=0.09; p=0.06).

TABLE 5
The effect of BGP on cytokine secretion by OVA-specific
stimulated splenocytes of OVA-sensitized mice ex vivo

| | | IFN-γ | IL-2 |
|---|---|---|---|
| | n | (pg/mL) | (pg/mL) |
| OVA-oil | 9 | 115 ± 53 | 58.4 ± 16.3 |
| OVA-BGP | 9 | 155 ± 139 | 60.2 ± 17.7 |
| OVA-Pred | 6 | 110 ± 50 | 66.9 ± 20.9 |
| PBS-oil | 6 | 138 ± 28 | 39.6 ± 13.5* |

| | | IL-4 | IL-5 | IL-13 |
|---|---|---|---|---|
| | n | (pg/mL) | (ng/mL) | (pg/mL) |
| OVA-oil | 9 | 36.8 ± 15.6 | 3.92 ± 1.30 | 430 ± 60 |
| OVA-BGP | 9 | 25.3 ± 12.5 | 2.93 ± 1.51 | 297 ± 12* |
| OVA-Pred | 6 | 33.4 ± 14.5 | 2.12 ± 1.85 | 322 ± 118 |
| PBS-oil | 6 | 34.5 ± 14.5 | 0.15 ± 0.10** | 131 ± 12** |

Note 1:
Each value represents mean ± SD
Note 2:
*p < 0.05;
**p < 0.01;
#0.05 p < 0.1 is the results of comparison with the control group by Student's t-test.

### Example 7

### Total Amount of IgE, IgG, IgA, and IgM in Serum

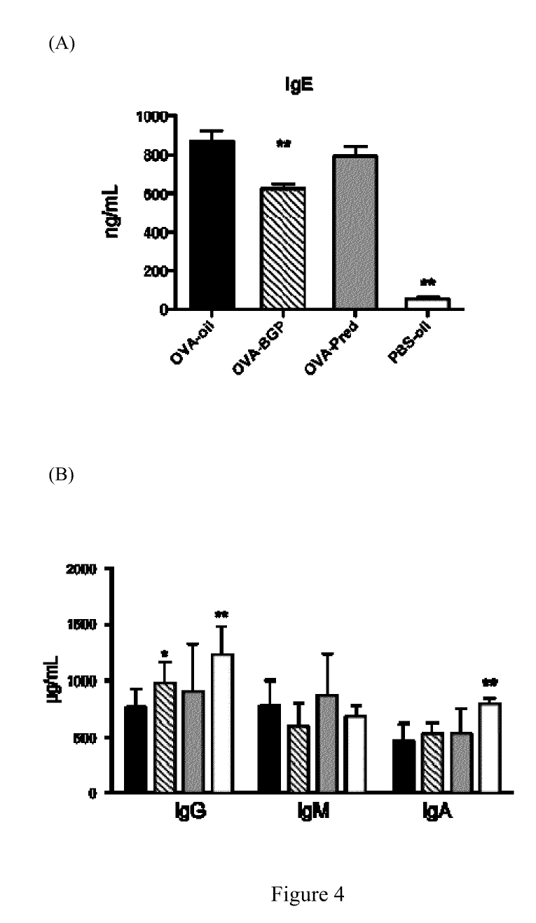

Referring to FIGS. 4A-4B, total amount of serum IgE, IgG, IgA and IgM in mouse model fed with BGP and CLN were exhibited. As compared to the OVA-oil and PBS-oil group, total serum IgE concentration was significantly higher and total IgG and IgA concentration was lower in mice fed with BGP and CLN. The results demonstrated that feeding B GP diet could reduce total serum IgE concentration and increase total serum IgG concentration.

### Example 8

### Expression of PPAR-α mRNA in the Lung Tissue

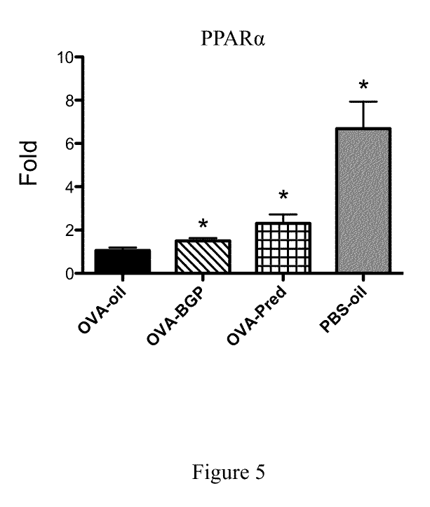

Effect of diet treatment on PPAR-α gene expression in lung tissue was exhibited in FIG. 5. As compared to the OVA-oil group, higher level of PPAR-α gene expression was observed in PBS-oil group, suggesting allergic immunity induced by sensitization process had decreased PPAR-α gene expression in the lung while mice fed with BGP showed enhanced PPAR-α gene expression.

The results of the Examples of the present invention demonstrated that airway resistance was alleviated in mice fed with BGP and CLN. Regarding to the changes of cell number of leukocytes in the BALF, total leukocyte counts was significant less and infiltration of eosinophils, basophils, neutrophils was reduced in BGP treated mice group. Administration of BGP could decrease the level of Th2 Cytokines (including IL-4, IL-5, IL-13, and IL-6), eotaxin, PEG2 inflammatory factors in airway, Furthermore, Administration of BGP also could decrease the total amount of IgE while increase IgG level and expression of PPARα mRNA in the lung. These results demonstrated that ingestion of BGP could alleviate airway inflammation in mouse model of allergenic asthma.

To summarize description above, bitter melon can be developed to as a health food to prevent asthma, or to alleviate the various symptoms of allergic asthma. The bitter melon also can be developed as a food composition; a single ingredient; a food material or ingredient that containing an effective dosage of the present invention; or an additive for combination with healthy ingredient, a food material, or combination of both nutraceutical ingredient and food material, to alleviate symptoms of allergic asthma.

From the abovementioned examples, the present invention provides a use of bitter melon composition for the manufacture of a medicament for alleviating allergic asthma. The application of the present invention can be prepared and produced according known methods but not limited thereof.

What is claimed is:

1. A method for alleviating allergic asthma and allergic response, comprising administrating a bitter melon composition to a subject suffering from allergic asthma, wherein the bitter melon composition comprises at least an effective amount of conjugated linolenic acid (CLN).

2. The method of claim 1, wherein the bitter melon composition is freeze dried powder made of whole bitter melon fruit.

3. The method of claim 2, wherein the bitter melon composition is mixed with at least one edible ingredient and then administered to the subject.

4. The method of claim 3, wherein the effective amount of the conjugated linolenic acid is preferred to be higher than 1% (W/V).

5. The method of claim 1, wherein the bitter melon composition is administered to the subject without interfering with the subject's growth.

6. The method of claim 1, wherein the bitter melon composition alleviates the subject's airway hyperresponsiveness (AHR).

7. The method of claim 1, wherein the bitter melon composition reduces a level of interleukin-4 (IL-4), interleukin-5 (IL-5), interleukin-13 (IL-13), interleukin-6 (IL-6), eotaxin, prostaglandin E₂ (PGE₂), or mixture thereof in the subject's lung.

8. The method of claim 1, wherein the bitter melon composition decreases a number of eosinophils, basophils, neutrophils or mixture thereof in the subject's lung.

9. The method of claim 1, wherein the bitter melon composition does not affect the propagation of ovalbumin-specific splenocytes in the subject.

10. The method of claim 1, wherein the bitter melon composition decreases the secretion of IL-13 of ovalbumin-specific challenged splenocytes.

11. The method of claim 1, wherein the bitter melon composition decreases total level of immunoglobulin E (IgE) of the subject.

12. The method of claim 1, wherein the bitter melon composition increases a level of immunoglobulin G of the subject.

13. A bitter melon composition for alleviation of allergic asthma, comprising at least an effective amount of conjugated linolenic acid, wherein the bitter melon composition alleviates a subject's allergic asthma symptom.

14. The bitter melon composition of claim 13 which is freeze dried powder made of whole bitter melon fruit.
